(19) United States
(12) Patent Application Publication
(10) Pub. No.: US 2024/0307477 A1
(43) Pub. Date: Sep. 19, 2024

(54) COMPOSITION FOR PREVENTING OR TREATING LIVER FIBROSIS INCLUDING SANGUISORBA OFFICINALIS EXTRACT AS ACTIVE INGREDIENT

(71) Applicant: KOREA INSTITUTE OF SCIENCE AND TECHNOLOGY, Seoul (KR)

(72) Inventors: Chu Won NHO, Gangneung-si (KR); Myungsuk KIM, Gangneung-si (KR); Yunseong NAM, Gangneung-si (KR)

(73) Assignee: KOREA INSTITUTE OF SCIENCE AND TECHNOLOGY, Seoul (KR)

(21) Appl. No.: 18/441,653

(22) Filed: Feb. 14, 2024

(30) Foreign Application Priority Data
Feb. 14, 2023 (KR) 10-2023-0019605

(51) Int. Cl. A61K 36/739; A61K 9/00; A61K 31/704; A61P 1/16

(52) U.S. Cl. CPC A61K 36/739; A61K 9/0056; A61K 31/704; A61P 1/16; A61K 2236/33; A61K 2236/331; A61K 2236/333

(57) ABSTRACT

The present invention relates to a composition for preventing or treating liver fibrosis, which includes a Sanguisorba officinalis extract as an active ingredient, and more particularly, to a composition for preventing or treating liver fibrosis, which includes a Sanguisorba officinalis extract or ziyuglycoside. The Sanguisorba officinalis extract or ziyuglycoside of the present invention inhibits liver fibrosis and the expression of a liver fibrosis-related gene, so it may be effectively used in the composition for preventing or treating liver fibrosis.

### CROSS-REFERENCE TO RELATED APPLICATION

This application claims priority to and the benefit of Korean Patent Application No. 10-2023-0019605, filed on Feb. 14, 2023, the disclosure of which is incorporated herein by reference in its entirety.

### REFERENCE TO AN ELECTRONIC SEQUENCE LISTING

The contents of the electronic sequence listing (“NewApp_0212090008_SeqListAsFiledXML.xml”; Size is 30 kilobytes and it was created on Feb. 13, 2024) is herein incorporated by reference in its entirety.

### BACKGROUND

### 1. Field of the Invention

The present invention relates to a composition for preventing or treating liver fibrosis, which includes a Sanguisorba officinalis extract as an active ingredient, and more particularly, to a composition for preventing or treating liver fibrosis, which includes a Sanguisorba officinalis extract or ziyuglycoside.

### 2. Discussion of Related Art

Liver fibrosis is a part of the bioadaptive response accompanying toxic materials, or various infectious, immune, and metabolic diseases, and is a condition in which collagen accumulation occurs without normal restoration of damaged liver tissue, like fibrous tissue. It is characterized by excessive accumulation of extracellular matrix, collagen, glycoproteins such as fibronectin and laminin, and proteoglycans, and the main component of the accumulated product is collagen, particularly type I collagen (Olaso E et. al., J. Hepatol., 29:836-847, 1998).

Liver fibrosis is known to be reversible, formed of thin fibrils, and have no nodules, so it may be possible to recover to normal once the cause of liver damage disappears. However, when the fibrosis process continues repeatedly, the exchange and bonding between extracellular matrices (ECMs) increase, which may lead to irreversible cirrhosis with the formation of thick fibrils and regenerative nodules.

Currently, as materials for inhibiting liver fibrosis, penicillamine, 16,16-dimethyl prostigladin E2, biphenyl dimethyldicarboxylic acid, colchicine, glucocorticoid, malotilate, y-interferon, pentoxifylline, pyridine-2,4-dicarboxylic-diethylamide, and pyridine-2,4-dicarboxylic-di(2-methoxyethyl)amide have been developed, but when applied clinically, they have problems with weak effects or severe side effects (Korean Patent No. 10-1632839).

Meanwhile, Jiyu (Sanguisorba officinalis (root)) is the root of Sanguisorba officinalis or Sanguisorba officinalis L. var. longifolis (Bert.) Yu et Li belonging to the genus Sanguisorba (family: Rosaceae). Jiyu is an important folk medicine used for diarrhea, colitis, bleeding, sores, burns, etc. and widely used to stop various types of bleeding due to its particularly strong hemostatic effect. The antiviral efficacy and skin disease treatment efficacy of the Sanguisorba officinalis extract have also been reported (Korean Laid-Open Publication No. 10-2006-0102621; and Korean Laid-Open Publication No. 10-2017-0120509).

### SUMMARY OF THE INVENTION

Therefore, as a result of intensively trying to develop an effective therapeutic agent for liver fibrosis in the present invention, it was confirmed that liver fibrosis and the expression of a liver fibrosis-related gene were inhibited by a Sanguisorba officinalis extract or ziyuglycoside, and thus the present invention was completed.

Accordingly, the present invention is directed to providing a pharmaceutical composition for preventing or treating liver fibrosis, which includes a Sanguisorba officinalis extract or ziyuglycoside as an active ingredient.

The present invention is also directed to providing a health functional food composition for preventing or treating liver fibrosis, which includes a Sanguisorba officinalis extract or ziyuglycoside as an active ingredient.

To achieve the above-described purposes, according to an aspect of the present invention, there is provided a pharmaceutical composition for preventing or treating liver fibrosis, which includes a Sanguisorba officinalis extract or ziyuglycoside as an active ingredient.

According to another aspect of the present invention, there is provided a health functional food composition for preventing or treating liver fibrosis, which includes a Sanguisorba officinalis extract or ziyuglycoside as an active ingredient.

In one exemplary embodiment of the present invention, the Sanguisorba officinalis extract may be extracted with at least one solvent selected from distilled water (or water), lower alcohols having 1 to 4 carbon atoms, glycerin, ethyl acetate, acetone, butylene glycol, propylene glycol, dichloromethane, chloroform, ethylether, butylene glycol, hexane, and a mixed solvent thereof.

In another exemplary embodiment of the present invention, the ziyuglycoside may be ziyuglycoside I or ziyuglycoside II.

In still another exemplary embodiment of the present invention, the Sanguisorba officinalis extract or ziyuglycoside may inhibit liver fibrosis through the inhibition of the expression of any one or more liver fibrosis-related genes selected from the group consisting of alpha-1 type I collagen (COL1A1), alpha-1 chain of type III collagen (COL3A1), transforming growth factor beta 1 (TGF-β1), actin alpha 2, smooth muscle (ACTA2) and TIMP metallopeptidase inhibitor 1 (TIMP1) genes.

In yet another exemplary embodiment of the present invention, the Sanguisorba officinalis extract may be included at 1 to 100 μg/mL in the composition, and the ziyuglycoside may be included at 1 to 100 μM in the composition.

### BRIEF DESCRIPTION OF THE DRAWINGS

The above and other objects, features and advantages of the present invention will become more apparent to those of ordinary skill in the art by describing exemplary embodiments thereof in detail with reference to the accompanying drawings, in which:

FIG. 1 shows a set of data that confirm the effects of inhibiting the expression of liver fibrosis-related genes, such as actin alpha 2, smooth muscle (ACTA2) gene, alpha-1 type I collagen (COL1A1) gene, and TIMP metallopeptidase inhibitor 1 (TIMP1) gene by a Sanguisorba officinalis extract; in this drawing, Control indicates an untreated control group, and SO indicates a Sanguisorba officinalis extract (SO1=Sanguisorba officinalis extract 1 μg/mL, SO5=Sanguisorba officinalis extract 5 μg/mL, and SO10=Sanguisorba officinalis extract 10 μg/mL);

FIG. 2 shows a set of data that confirm the effects of inhibiting the expression of liver fibrosis-related genes, such as (A) actin alpha 2, smooth muscle (ACTA2) gene, (B) alpha-1 type I collagen (COL1A1) gene, and (C) TIMP metallopeptidase inhibitor 1 (TIMP1) gene by ziyuglycoside. In this drawing, Control indicates an untreated control group;

FIG. 3 shows a set of data that confirm the effects of inhibiting the expression of liver fibrosis-related genes, such as (A) actin alpha 2, smooth muscle (ACTA2) gene, (B) alpha-1 type I collagen (COL1A1) gene, and (C) TIMP metallopeptidase inhibitor 1 (TIMP1) gene by a Sanguisorba officinalis extract according to an ethanol content;

FIG. 4 shows a set of images of observations of the changes in liver tissue by a Sanguisorba officinalis extract in liver fibrosis-induced mice; in this drawing, Control indicates a normally fed group, and OCA indicates an obeticholic acid-fed group (positive control); SO25 indicates a group fed with 25 mg/kg of the Sanguisorba officinalis extract, and SO100 indicates a group fed with 100 mg/kg of the Sanguisorba officinalis extract;

FIG. 5 shows data that confirm the effect of promoting the expression of peroxisome proliferator-activated receptor alpha (Ppara) gene, which is a fatty acid oxidation factor, in liver tissue by a Sanguisorba officinalis extract in liver fibrosis-induced mice;

FIG. 6 shows data that confirm the effect of inhibiting the expression of lipoprotein lipase (Lp1) gene in liver tissue by a Sanguisorba officinalis extract in liver fibrosis-induced mice;

FIG. 7 shows data that confirm the effect of inhibiting the production of malondialdehyde (MDA), which is a lipid peroxide, in liver tissue by a Sanguisorba officinalis extract in liver fibrosis-induced mice;

FIG. 8 shows data that confirm the effect of promoting the expression of Superoxide Dismutase 1 (Sod1) gene, which is an antioxidation-related enzyme, by a Sanguisorba officinalis extract in liver fibrosis-induced mice;

FIG. 9 shows data that confirm the effect of promoting the expression of catalase (Cat) gene, which is an antioxidation-related enzyme, in liver tissue by a Sanguisorba officinalis extract in liver fibrosis-induced mice;

FIG. 10 shows data that confirm the effect of promoting the expression of Glutathione Peroxidase 1 (Gpx1) gene, which is an antioxidation-related enzyme, in liver tissue by a Sanguisorba officinalis extract in liver fibrosis-induced mice;

FIG. 11 shows a set of images of fibrotic tissue stained with Sirius red to confirm the degree of inhibiting liver fibrosis in liver tissue by a Sanguisorba officinalis extract in liver fibrosis-induced mice;

FIG. 12 shows data that confirm the effect of inhibiting a hydroxyl proline content to confirm a liver fibrosis level in liver tissue by a Sanguisorba officinalis extract in liver fibrosis-induced mice;

FIG. 13 shows data that confirm the effect of inhibiting the expression of alpha-1 type I collagen (COL1A1) gene, which is a liver fibrosis-related gene, in liver tissue by a Sanguisorba officinalis extract in liver fibrosis-induced mice;

FIG. 14 shows data that confirm the effect of inhibiting the expression of alpha-1 chain of type III collagen (COL3 A1) gene, which is a liver fibrosis-related gene, in liver tissue by a Sanguisorba officinalis extract in liver fibrosis-induced mice; and

FIG. 15 shows data that confirm the effect of inhibiting the expression of transforming growth factor beta 1 (TGF-β1) gene, which is a liver fibrosis-related gene, in liver tissue by a Sanguisorba officinalis extract in liver fibrosis-induced mice.

### DETAILED DESCRIPTION OF EXEMPLARY EMBODIMENTS

Hereinafter, the present invention will be described in detail.

In one aspect, the present invention relates to a pharmaceutical composition for preventing or treating liver fibrosis, which includes a Sanguisorba officinalis extract or ziyuglycoside as an active ingredient.

In another aspect, the present invention relates to a health functional food composition for preventing or treating liver fibrosis, which includes a Sanguisorba officinalis extract or ziyuglycoside as an active ingredient.

In the present invention, the Sanguisorba officinalis extract may be prepared using a conventional solvent according to a conventional method known in the art, that is, under common temperature and pressure conditions. The Sanguisorba officinalis extract is preferably extracted using at least one solvent selected from the group consisting of distilled water (or water), lower alcohols having 1 to 4 carbon atoms, glycerin, ethyl acetate, acetone, butylene glycol, propylene glycol, dichloromethane, chloroform, ethylether, butylene glycol, hexane, and a mixed solvent thereof, and more preferably, using ethanol or water. However, the present invention is not limited thereto.

In the present invention, the ziyuglycoside may be ziyuglycoside I or ziyuglycoside II.

In the present invention, the Sanguisorba officinalis extract or ziyuglycoside may inhibit liver fibrosis by inhibiting the expression of any one or more liver fibrosis-related genes selected from the group consisting of alpha-1 type I collagen (COL1A1) gene, alpha-1 chain of type III collagen (COL3A1) gene, transforming growth factor beta 1 (TGF-β1) gene, actin alpha 2, smooth muscle (ACTA2) gene, and TIMP metallopeptidase inhibitor 1 (TIMP1) gene.

In the present invention, the composition may further include a material or extract having an effect of preventing or treating liver fibrosis other than a Sanguisorba officinalis extract.

In the present invention, the Sanguisorba officinalis extract may be included at 1 to 100 μg/mL, preferably 1 to 50 μg/mL, and more preferably 1 to 10 μg/mL in the composition, and the ziyuglycoside may be included at 1 to 100 μM, preferably 1 to 50 μM, and more preferably 1 to 10 μM in the composition. When the extract is included at less than 1 μg/mL or 1 μM, the effect of preventing or treating liver fibrosis may be insignificant, and when the extract is included at more than 100 μg/mL or 100 μM, cytotoxicity may be shown.

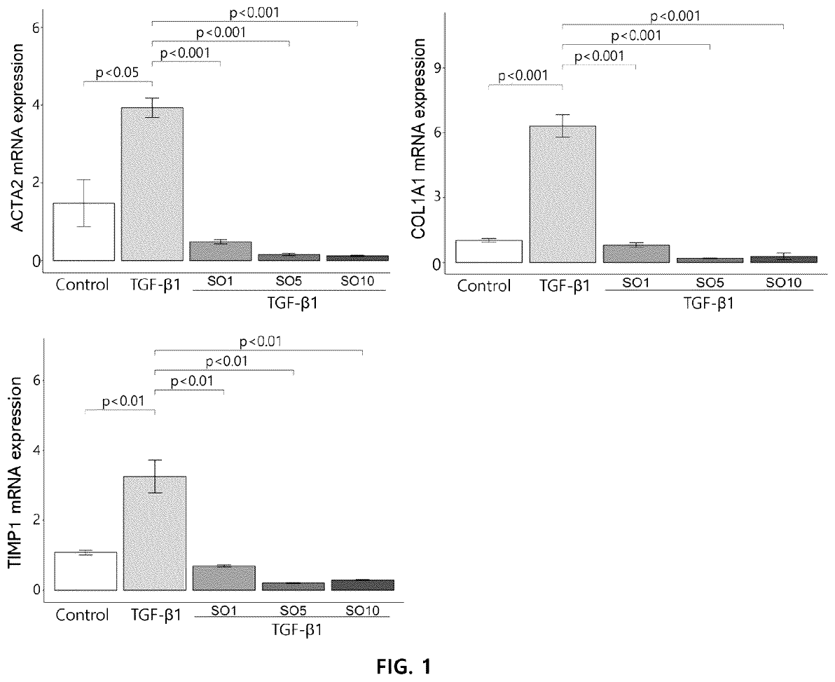

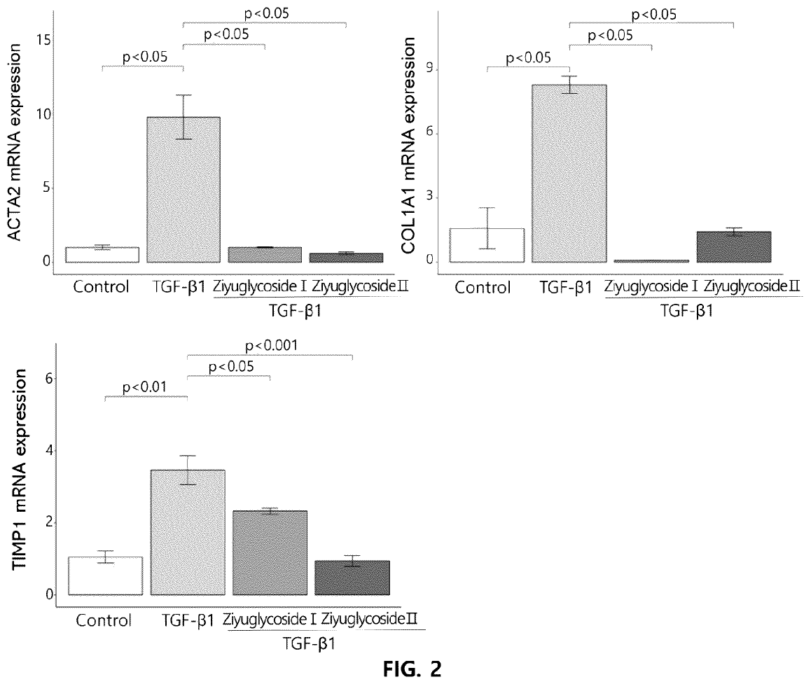

In an exemplary embodiment of the present invention, as a result of confirming the efficacy of inhibiting the expression of liver fibrosis-related genes by a Sanguisorba officinalis 95% ethanol extract, ziyuglycoside I, and ziyuglycoside II, it was confirmed that mRNA expression of the actin alpha 2, smooth muscle (ACTA2) gene, alpha-1 type I collagen (COL1A1) gene and TIMP metallopeptidase inhibitor 1 (TIMP1) gene is inhibited by the Sanguisorba officinalis extract and ziyuglycoside (FIGS. 1 to 2).

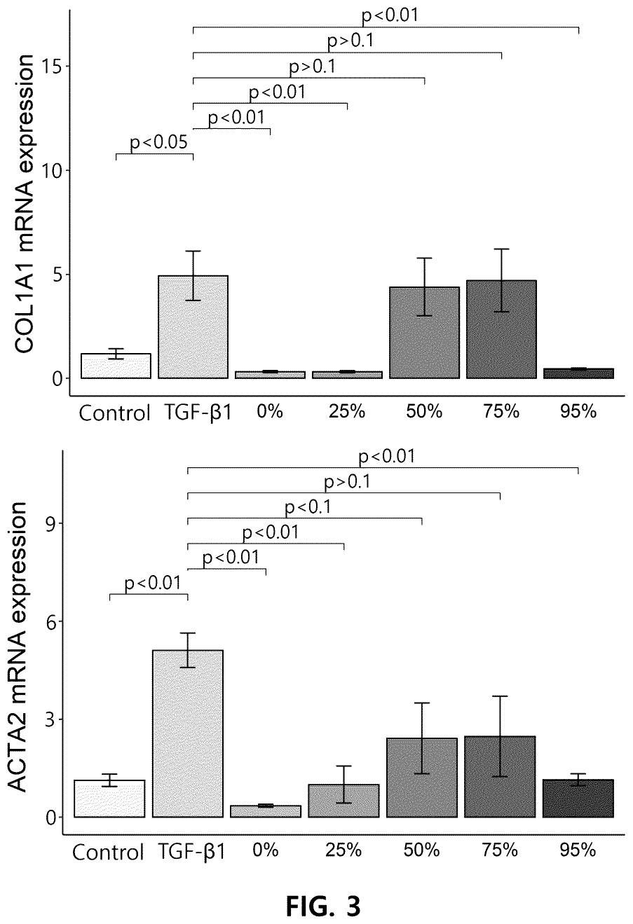

In another exemplary embodiment of the present invention, as a result of confirming the effect of a Sanguisorba officinalis extract in inhibiting a liver fibrosis-related gene according to an ethanol content, it was confirmed that the expression of a liver fibrosis-related gene is effectively inhibited by the treatment of a 0% ethanol (100% water) Sanguisorba officinalis extract, a 25% ethanol Sanguisorba officinalis extract, and a 95% ethanol Sanguisorba officinalis extract (FIG. 3).

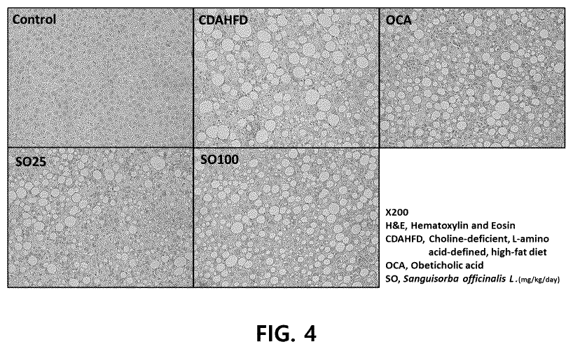

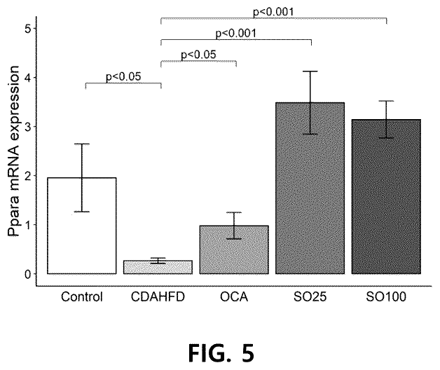

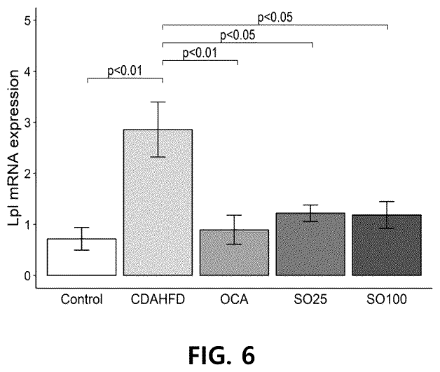

In still another exemplary embodiment of the present invention, as a result of administering a Sanguisorba officinalis extract to liver fibrosis-induced mice, it was confirmed that lipid accumulation in liver tissue is reduced (FIG. 4), the expression of peroxisome proliferator-activated receptor alpha (Ppara) gene, which is a fatty acid oxidation factor, is promoted, and the expression of lipoprotein lipase (Lp1) gene is inhibited (FIGS. 5 and 6).

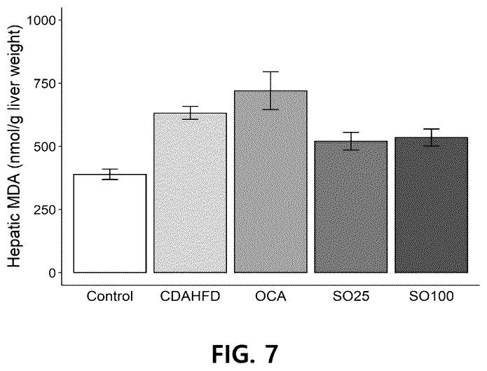

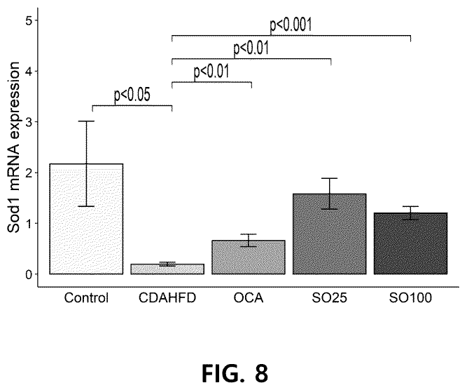

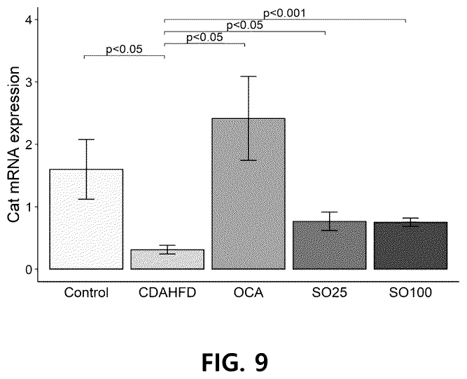

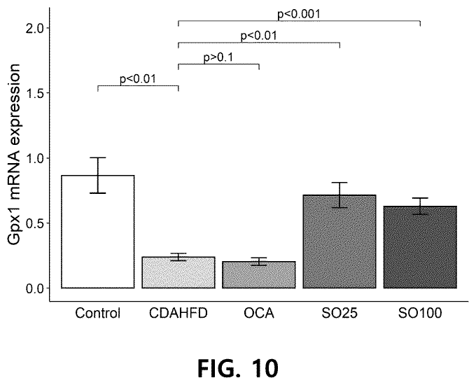

In addition, it was confirmed that, by the Sanguisorba officinalis extract, the generation of malondialdehyde (MDA), which is a lipid peroxide, in liver tissue is inhibited (FIG. 7), and the expression of superoxide dismutase 1 (Sod1) gene, catalase (Cat) gene, and glutathione peroxidase 1 (Gpx1) gene, which are antioxidation-related enzymes, is promoted (FIGS. 8 to 10).

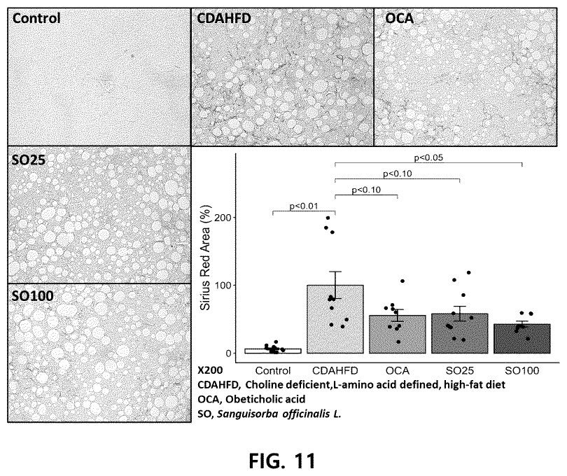

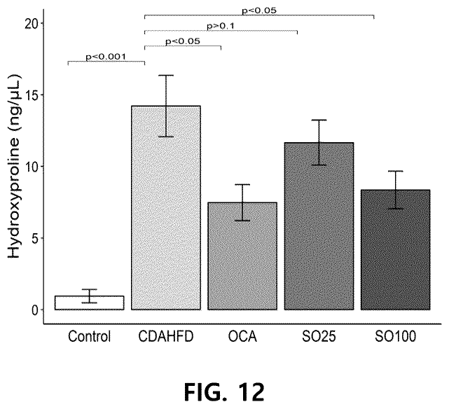

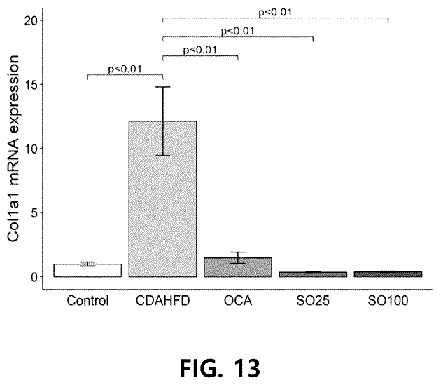

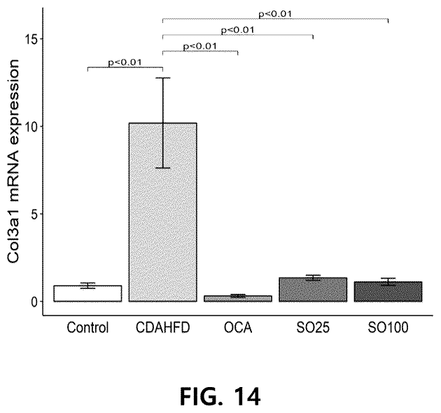

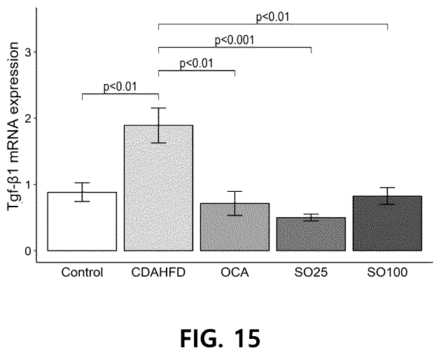

Further, it was confirmed that liver fibrosis is inhibited by the Sanguisorba officinalis extract in liver fibrosis-induced mice (FIGS. 11 and 12), and the expression of liver fibrosis-related genes, such as alpha-1 type I collagen (COL1A1) gene, alpha-1 chain of type III collagen (COL3A1) gene, and transforming growth factor beta 1 (TGF-β1) gene, is inhibited (FIGS. 13 to 15).

That is, it was confirmed that the Sanguisorba officinalis extract or ziyuglycoside of the present invention inhibits liver fibrosis and the expression of a liver fibrosis-related gene, so it can be effectively used in a composition for preventing or treating liver fibrosis.

The pharmaceutical composition of the present invention may be used by being formulated in various forms according to conventional methods. For example, the pharmaceutical composition of the present invention may be formulated in an oral formulation such as a powder, a granule, a tablet, a capsule, a suspension, an emulsion, or a syrup, or may be formulated in an external preparation, a suppository, or a sterile injectable solution. The pharmaceutical composition of the present invention may further include a pharmaceutically acceptable carrier, excipient and diluent depending on a formulation. In addition, it may be used by being formulated in the form of a powder, a granule, a tablet, a capsule, a suspension, an emulsion, a syrup, or an external preparation such as an aerosol, or a sterile injectable solution according to a conventional method.

As the carrier, excipient and diluent, lactose, dextrose, sucrose, sorbitol, mannitol, xylitol, erythritol, maltitol, starch, acacia gum, alginate, gelatin, calcium phosphate, calcium silicate, cellulose, methyl cellulose, microcrystalline cellulose, polyvinyl pyrrolidone, methylhydroxy benzoate, propylhydroxybenzoate, talc, magnesium stearate, or mineral oil is used. When preparing or formulating the pharmaceutical composition, a generally used diluent or excipient such as a filler, an extender, a binder, a wetting agent, a disintegrant, or a surfactant is used.

Solid preparations for oral administration include tablets, pills, powders, granules, and capsules, and such solid preparations are prepared by mixing at least one excipient, such as starch, calcium carbonate, sucrose, lactose, or gelatin, with the composition. Aside from simple excipients, lubricants such as magnesium stearate, talc, etc. are further used. As liquid preparations for oral administration, a suspension, a liquid for internal use, an emulsion, or a syrup may be used, and a generally used simple diluent such as water or liquid paraffin, as well as various types of excipients, for example, a wetting agent, a sweetener, a fragrance and a preservative, may be included. Preparations for parenteral administration include sterilized aqueous solutions, non-aqueous solvents, suspensions, emulsions, lyophilized formulations, and suppositories. As the non-aqueous solvent or suspension, propylene glycol, polyethylene glycol, a vegetable oil such as olive oil, or an injectable ester such as ethyl oleate may be used. As a suppository base, Witepsol, Macrogol, Tween 61, cacao butter, laurinum, or glycerogelatin may be used.

The term “administration” used in the present invention refers to providing a pharmaceutical composition of the present invention to a subject by any suitable method. The pharmaceutical composition of the present invention may be administered at a therapeutically effective amount, which means an amount of an active ingredient or pharmaceutical composition that induces a biological or medical reaction in a tissue system, animal, or human considered by a researcher, veterinarian, physician, or other clinical trials, that is, an amount that induces the alleviation of symptoms of a treated disease or disorder. It is apparent to those of ordinary skill in the art that the therapeutically effective dosage and number of administrations of the pharmaceutical composition of the present invention will vary depending on a desired effect. Therefore, the optimal dosage to be administered may be easily determined by one of ordinary skill in the art, and may be adjusted according to various factors including the type of disease, the severity of the disease, the contents of an active ingredient and other ingredients contained in a composition, the type of formulation, a patient's age, body weight, general health condition, sex, and diet, the time of administration, an administration route, the secretion rate of a composition, the duration of treatment, and a concurrent drug. The pharmaceutical composition of the present invention may be administered to a subject via various routes. For example, the pharmaceutical composition of the present invention may be administered intravenously, intraperitoneally, intramuscularly, intraarterially, buccally, intracardiacally, intramedullarily, intrathecally, transdermally, enterally, subcutaneously, sublingually, or topically, but the present invention is not limited thereto. The pharmaceutical composition of the present invention may be administered at 1 to 10,000 mg/kg/day at once or in divided portions.

The health functional food composition of the present invention may be used as a health functional food, food additive, or dietary supplement. When using the extract of the present invention as a food additive, it may be used alone, or appropriately used by a conventional method of mixing with another food or food component.

In addition, the mixing amount of the health functional food composition may be suitably modified depending on the purpose of use (for prevention, health, or curative treatment). As a specific example, when manufacturing food or beverages, the extract of the present invention is added in an amount of 15 wt % or less, and preferably 10 wt % or less, based on the raw material. However, when ingested for a long period for health and hygiene or health control, it may be added in an amount below this range. Since there is no problem in terms of safety, the active ingredient may also be used in an amount above this range.

There is no particular limitation on the type of food, but examples of food to which the extract of the present invention may include meat, sausages, bread, chocolate, candy, snacks, confectionery, pizza, ramen, other noodles, gum, dairy products including ice cream, various kinds of soups, beverages, tea, drinks, alcoholic beverages, and vitamin complexes, and include all health foods in a common sense.

When the health functional food composition of the present invention is manufactured in a beverage, it may further include additional components such as various flavoring agents or natural carbohydrates, like conventional beverages. As the natural carbonates, monosaccharides such as glucose and fructose; disaccharides such as maltose and sucrose; natural sweeteners such as dextrin and cyclodextrin; and synthetic sweeteners such as saccharin and aspartame may be used. The natural carbohydrate may be included at 0.01 to 10 wt %, and preferably 0.01 to 0.1 wt % with respect to the total weight of the food composition of the present invention.

The health functional food composition of the present invention may include various nutrients, vitamins, electrolytes, flavors, colorants, pectic acid and its salts, alginic acid and its salts, organic acids, protective colloidal thickeners, pH adjusters, stabilizers, preservatives, glycerin, alcohols, and carbonating agents, natural fruit juice, or pulp for producing fruit juice drinks or vegetable drinks, but the present invention is not limited thereto. These components may be used independently or in combination. The ratio of the above additives is not greatly limited and is preferably within the range of 0.01 to 0.1 wt % with respect to the total weight of the food composition of the present invention.

Hereinafter, preferred examples are presented to allow the present invention to be better understood. However, the following examples are provided only so that the present invention may be more easily understood, and the content of the present invention is not limited by the following examples.

### Example 1

Preparation of Sanguisorba officinalis Extract

In the present invention, the optimal extraction conditions including liver fibrosis inhibitory effect were established from a Sanguisorba officinalis extract. The extraction was obtained by purchasing Sanguisorba officinalis at Gyeongdong Market, cutting it into pieces of appropriate size, cold-extracting the pieces by adding 1 kg of Sanguisorba officinalis and 5 L of 95% (v/v) ethanol to an extraction container, and filtering the extracted product through a filter paper. The extraction process was repeated three times, and a solvent was then concentrated under reduced pressure and dried to obtain 80 g of ethanol extract. The ethanol extract was used in cell and animal experiments.

In addition, to establish the optimal extraction conditions of Sanguisorba officinalis, an extract was obtained by cold extraction by mixing 0% (v/v) (100% water), 25% (v/v), 50% (v/v), 70% (v/v), or 95% (v/v) ethanol to 10 g of the Sanguisorba officinalis pieces of appropriate size, and filtering the resulting product through a filter paper using an extraction solvent.

### Example 2

Confirmation of Effects of Inhibiting Liver Fibrosis-Producing Gene by Sanguisorba officinalis Extract and Ziyuglycoside (Ziyuglycoside I, Ziyuglycoside II) in in Hepatic Stellate Cells

As hepatic stellate cells, human immortalization hepatic stellate cells (human LX-2) were given and used. The hepatic stellate cells were cultured in Dulbecco's modified Eagle's medium (DMEM; Invitrogen, US) containing 10% fetal bovine serum (FBS), penicillin (100 U/mL) and streptomycin (100 mg/mL) at 37° C. and in a 5% CO₂ condition.

To measure the hepatic anti-fibrosis effect of the Sanguisorba officinalis extract (1, 5 or 10 μg/mL) and ziyuglycoside (5 μM) in the LX-2 cells, a real time-quantitative polymerase chain reaction (RT-qPCR) was performed.

First, the hepatic stellate cells were treated with each of the Sanguisorba officinalis extract (1, 5, or 10 μg/mL) and the ziyuglycoside (5 μM) along with 5 ng/mL of a liver fibrosis-inducing material TGF-β and cultured for 24 hours. Afterward, the cells were washed with PBS, total RNA was then extracted according to the manual of an RNA extraction kit (GeneAll RNA isolation kit, Korea), and cDNA was synthesized from the extracted RNA. PCR analysis was performed under user recommended cycling conditions of an analyzer (Applied Biosystems 7900 HT thermal cycler, Applied Biosystems), and to confirm the expression level of each mRNA, PCR was performed using the primers in Table 1 below, and the fold change value for the control was calculated using GAPDH as an endogenous control, and a relative expression level was compared.

TABLE 1
Primer sequences

| Gene | Primer sequence | | SEQ ID NO: |
|---|---|---|---|
| COL1A1 | Forward | ATG GAG CTC CTG GTC | SEQ ID NO: |
| | primer | AGA T | 1 |
| | Reverse | GTA GCA CCA TCA TTT | SEQ ID NO: |
| | primer | CCA CG | 2 |
| ACTA2 | Forward | GCC AAG CAC TGT CAG | SEQ ID NO: |
| | primer | GAA | 3 |
| | Reverse | ATT GTC ACA CAC CAA | SEQ ID NO: |
| | primer | GGC A | 4 |
| TIMP1 | Forward | CTC TGA AAA GGG CTT | SEQ ID NO: |
| | primer | CCA GTC | 5 |
| | Reverse | AGG ATT CAG GCT ATC | SEQ ID NO: |
| | primer | TGG GAC | 6 |
| GAPDH | Forward | CAG CCG CAT CTT CTT | SEQ ID NO: |
| | primer | TTG CG | 7 |
| | Reverse | TCC GTT GAC TCC GAC | SEQ ID NO: |
| | primer | CTT CA | 8 |

As a result, as shown in FIGS. 1 and 2, it was confirmed that the gene expression of fibrosis factors such as COL1A1 gene, ACTA2 gene and TIMP1 gene, increased by TGF-β treatment, was reduced by the Sanguisorba officinalis extract and ziyuglycoside treatment.

### Example 3

Confirmation of Effect of Inhibiting the Expression of Liver Fibrosis-Generating Gene Per Ethanol Content by Sanguisorba officinalis Extract in Hepatic Stellate Cells

To measure the hepatic anti-fibrosis effect of the Sanguisorba officinalis extract according to the ethanol content in LX-2 cells, real time-quantitative polymerase chain reaction (RT-qPCR) was performed.

First, the hepatic stellate cells were treated with 20 μg/mL each of the Sanguisorba officinalis extracts (0, 25, 50, 70, or 95% ethanol extract) by content of ethanol prepared in Example 1, together with 5 ng/ml of the liver fibrosis-inducing material TGF-β, and cultured for 24 hours. Afterward, the cells were washed with PBS, total RNA was then extracted according to the manual of an RNA extraction kit (GeneAll RNA isolation kit, Korea), and cDNA was synthesized from the extracted RNA. PCR analysis was performed under user recommended cycling conditions of an analyzer (Applied Biosystems 7900 HT thermal cycler, Applied Biosystems), and to confirm the expression level of each mRNA, PCR was performed using the primers in Table 1 below, and the fold change value for the control was calculated using GAPDH as an endogenous control, and a relative expression level was compared.

As a result, as shown in FIG. 3, it was confirmed that the expression of a liver fibrosis-related gene such as COL1A1 gene or ACTA2 gene, which is a fibrosis factor increased by TGF-β treatment, is effectively inhibited by the treatment of the 0% ethanol (100% water) Sanguisorba officinalis extract, the 25% ethanol Sanguisorba officinalis extract, and the 95% ethanol Sanguisorba officinalis extract.

### Example 4

Confirmation of Effect of Inhibiting Lipid Accumulation in Liver Tissue by Sanguisorba officinalis Extract in Liver Fibrosis-Inducing Mice

C57BL/6J mice (male, 6-week-old) were obtained from DooYeol Biotech (Korea) and acclimated for 2 weeks, and then divided into five experimental groups (n=10 per group) by body weight. The experimental groups were fed with a choline-deficient, L-amino acid-defined, high-fat diet (CDA-HFD) for 10 weeks to induce liver fibrosis, and a normal control was fed with AIN76 diet (Research Diets). The dark-light cycle was maintained at 12 h-12 h intervals, and water was allowed to be consumed freely. The diets administered to the experimental groups, doses, and administration methods are shown in Table 2 below.

A test material was prepared in the form of a liquid preparation, which is a suspension, using 0.5% carboxymethylcellulose (CMC) and 4% sesame oil, and administered for 10 weeks. To compensate for the placebo effect and body weight reduction effect by administration stress of 0.5% CMC and 4% sesame oil, 0.5% CMC and 4% sesame oil were orally administered daily to a vehicle control. Obeticholic acid (OCA) was administered as a positive control, and the 95% ethanol Sanguisorba officinalis extract was administered at 25 mg/kg or 100 mg/kg for 10 weeks.

TABLE 2
Dosages and methods for drug administration
to liver fibrosis-inducing mice

| | | | Dosage | Adminis- |
|---|---|---|---|---|
| Experimental | | | and | tration |
| group | Feed | Test material | method | period |
| 1 | AIN76 diet | Vehicle | — | 10 weeks |
| 2 | CDA- | Vehicle | — | |
| 3 | HFD diet | Obeticholic | 15 mg/kg, | |
| | | acid | 1 day, once | |
| 4 | | Sanguisorba | 25 mg/kg, | |
| | | officinalis extract | 1 day, once | |
| 5 | | Sanguisorba | 100 mg/kg, | |
| | | officinalis extract | 1 day, once | |

After 10 weeks, as a result of observing mouse liver tissue by hematoxylin and eosin (H&E) staining, as shown in FIG. 4, while liver lipid accumulation was induced by CDA-HFD, lipid accumulation was confirmed to be reduced in the OCA-fed positive control group and the Sanguisorba officinalis extract-fed group, compared to the CDA-HFD-fed group.

### Example 5

Confirmation of Effect of Regulating Fatty Acid Oxidation Factor by Sanguisorba officinalis Extract in Liver Fibrosis-Induced Mice

After completing the administration of the Sanguisorba officinalis extract for a total of 10 weeks in the liver fibrosis-induced mice in Example 4, the mRNA expression levels of a fatty acid oxidation-related factor in liver tissue of all administered groups were confirmed using primers in Table 3 below.

TABLE 3
Primer sequences

| | | | SEQ ID |
|---|---|---|---|
| Gene | Primer sequence | | NO: |
| Ppara | Forward | GAA CTG ACG TTT GTG GCT | SEQ ID |
| | primer | GG | NO: 9 |
| | Reverse | GCT CTC TGT GTC CAC CAT | SEQ ID |
| | primer | GT | NO: 10 |
| Lpl | Forward | GTG GAC ATC GGA GAA CTG | SEQ ID |
| | primer | CT | NO: 11 |
| | Reverse | CCT CTC GAT GAC GAA GCT | SEQ ID |
| | primer | GG | NO: 12 |
| β- | Forward | CAT TGC TGA CAG GAT GCA | SEQ ID |
| actin | primer | GAA GG | NO: 13 |
| | Reverse | TGC TGG AAG GTG GAC AGT | SEQ ID |
| | primer | GAG G | NO: 14 |

As a result, as shown in FIGS. 5 and 6, compared to the AIN76-fed group (normal control), it was confirmed that the expression of the fatty acid oxidation factor Ppara gene was reduced and the expression of lipoprotein lipase (Lp1) gene was increased in the vehicle control fed with CDA-HFD, resulting in abnormality in lipid metabolism. On the other hand, it was confirmed that the OCA-fed positive control group and the Sanguisorba officinalis extract-fed group had higher Ppara gene expression and lower Lp1 gene expression, resulting in restoration of gene expression to a level similar to that of the normal control.

### Example 6

Confirmation of the Regulation in Oxidative Stress and Expression of Antioxidation-Related Enzyme by Sanguisorba officinalis Extract in Liver Fibrosis-Induced Mice

After completing the administration of the Sanguisorba officinalis extract for a total of 10 weeks in the liver fibrosis-induced mice in Example 4, the mRNA expression levels of an antioxidation-related enzyme in liver tissue of all administered groups were confirmed using primers in Table 4 below.

TABLE 4
Primer sequences

| Gene | Primer sequence | | SEQ ID NO: |
|---|---|---|---|
| Sod1 | Forward | GGG AAG CAT GGC | SEQ ID NO: 15 |
| | primer | GAT GAA AG | |
| | Reverse | GCC TTC TGC TCG | SEQ ID NO: 16 |
| | primer | AAG TGG AT | |
| Cat | Forward | CAA GAT TGC CTT | SEQ ID NO: 17 |
| | primer | CTC CGG GT | |
| | Reverse | ATG GTG TAG GAT | SEQ ID NO: 18 |
| | primer | TGC GGA GC | |
| Gpx1 | Forward | AGT CCA CCG TGT | SEQ ID NO: 19 |
| | primer | ATG CCT TC | |
| | Reverse | CCT CAG AGA GAC | SEQ ID NO: 20 |
| | primer | GCG ACA TT | |
| β- | Forward | CAT TGC TGA CAG | SEQ ID NO: 13 |
| actin | primer | GAT GCA GAA GG | |
| | Reverse | TGC TGG AAG GTG | SEQ ID NO: 14 |
| | primer | GAC AGT GAG G | |

To measure the effect of inhibiting MDA, which is the lipid peroxide of the Sanguisorba officinalis extract, MDA was measured and quantified using a thiobarbituric acid reactive substance (TBARS) assay (Biovision, Minneapolis, USA). Briefly, proteins separated from liver tissue with lysis buffer were reacted with a TBA regent at 65° C. for 45 minutes, and absorbance was then measured at 540 nm.

As a result, as shown in FIGS. 7 to 10, compared to a control fed with the AIN76 diet, it was confirmed that the vehicle control fed with CDA-HFD increased the lipid peroxide MDA, and the mRNA expression of antioxidation-related enzymes such as Sod1 gene, Cat gene, and Gpx1 gene was rapidly reduced. On the other hand, it was confirmed that the Sanguisorba officinalis extract-fed group significantly reduced the generation of MDA, and the mRNA expression of the antioxidation-related enzyme was increased, thereby reducing oxidative stress in the liver.

### Example 7

Confirmation of Liver Fibrosis Inhibitory Effect by Sanguisorba officinalis Extract in Liver Fibrosis-Induced Mice

After completing the administration of the Sanguisorba officinalis extract for a total of 10 weeks in the liver fibrosis-induced mice in Example 4, the liver tissue fragments of all administered groups were stained with Sirius red that stains fibrotic tissue, and liver fibrosis levels were confirmed by measuring a hydroxyl proline content in the liver tissue.

To measure a hydroxyl proline content, a hepatic collagen content was quantified by measuring hepatic hydroxyl proline (Biovision). Briefly, after homogenizing a liver tissue sample (20 mg) in an alkali hydrolysis buffer solution at 95° C. for 10 minutes, the hydroxyl proline concentration of a hydrolyte was measured at 550 nm. The amount was calculated by gram of liver tissue.

In addition, the mRNA expression levels of COL1A1 gene, COL3A1 gene, and TGF-β1 gene associated with liver fibrosis were confirmed using primers of Table 5 below.

TABLE 5
Primer sequences

| Gene | Primer sequence | | SEQ ID NO: |
|---|---|---|---|
| Colla1 | Forward | AGC ACG TCT GGT | SEQ ID NO: 21 |
| | primer | TTG GAG AG | |
| | Reverse | GAC ATT AGG CGC | SEQ ID NO: 22 |
| | primer | AGG AAG GT | |
| Col3a1 | Forward | GTG GAC ATT GGC | SEQ ID NO: 23 |
| | primer | CCT GTT TG | |
| | Reverse | AGT TGG TCA CTT | SEQ ID NO: 24 |
| | primer | GCA CTG GT | |
| Tgf- | Forward | GTG GCT GAA CCA | SEQ ID NO: 25 |
| β1 | primer | AGG AGA CG | |
| | Reverse | GTT TGG GGC TGA | SEQ ID NO: 26 |
| | primer | TCC CGT TG | |
| β- | Forward | CAT TGC TGA CAG | SEQ ID NO: 13 |
| actin | primer | GAT GCA GAA GG | |
| | Reverse | TGC TGG AAG GTG | SEQ ID NO: 14 |
| | primer | GAC AGT GAG G | |

As a result, as shown in FIGS. 11 and 12, compared to the control fed with the AIN76 diet, liver fibrosis developed rapidly in the vesicle control fed with CDA-HFD. On the other hand, a liver fibrosis area and a hydroxyl proline content were significantly inhibited in the OCA-fed positive control group and the Sanguisorba officinalis extract-fed group.

In addition, as shown in FIGS. 13 to 15, compared to the control fed with the AIN76 diet, it was confirmed that the mRNA expression of COL1A1 gene, COL3A1 gene, and TGF-β1 gene, associated with liver fibrosis, in the vehicle control fed with CDA-HFD was increased, but the mRNA expression of liver fibrosis-related factors was inhibited in the Sanguisorba officinalis extract-fed group.

Since it was confirmed that a Sanguisorba officinalis extract or ziyuglycoside of the present invention inhibits liver fibrosis and the expression of a liver fibrosis-related gene, it can be effectively used in a composition for preventing or treating liver fibrosis.

### SEQUENCE LISTING

Sequence list electronic file attached (C:\Users\keile\Desktop\PDPC224030.xml)

What is claimed is:

1. A pharmaceutical composition for preventing or treating liver fibrosis, which comprises a Sanguisorba officinalis extract or ziyuglycoside as an active ingredient.

2. The pharmaceutical composition of claim 1, wherein the Sanguisorba officinalis extract is extracted using at least one solvent selected from the group consisting of distilled water (or water), lower alcohols having 1 to 4 carbon atoms, glycerin, ethyl acetate, acetone, butylene glycol, propylene glycol, dichloromethane, chloroform, ethylether, butylene glycol, hexane, and a mixed solvent thereof.

3. The pharmaceutical composition of claim 1, wherein the ziyuglycoside is ziyuglycoside I or ziyuglycoside II.

4. The pharmaceutical composition of claim 1, wherein the Sanguisorba officinalis extract or ziyuglycoside inhibits liver fibrosis by inhibiting the expression of any one or more liver fibrosis-related genes selected from the group consisting of alpha-1 type I collagen (COL1A1) gene, alpha-1 chain of type III collagen (COL3A1) gene, transforming growth factor beta 1 (TGF-β1) gene, actin alpha 2, smooth muscle (ACTA2) gene, and TIMP metallopeptidase inhibitor 1 (TIMP1) gene.

5. The pharmaceutical composition of claim 1, wherein the Sanguisorba officinalis extract is included at 1 to 100 μg/mL in the composition, and the ziyuglycoside is included at 1 to 100 μM in the composition.

6. A health functional food composition for preventing or treating liver fibrosis, which comprises a Sanguisorba officinalis extract or ziyuglycoside as an active ingredient.

7. The health functional food composition of claim 6, wherein the Sanguisorba officinalis extract is extracted with at least one solvent selected from distilled water (or water), lower alcohols having 1 to 4 carbon atoms, glycerin, ethyl acetate, acetone, butylene glycol, propylene glycol, dichloromethane, chloroform, ethylether, butylene glycol, hexane, and a mixed solvent thereof.

8. The health functional food composition of claim 6, wherein the ziyuglycoside is ziyuglycoside I or ziyuglycoside II.

9. The health functional food composition of claim 6, wherein the Sanguisorba officinalis extract or ziyuglycoside inhibits liver fibrosis through the inhibition of the expression of any one or more liver fibrosis-related genes selected from the group consisting of alpha-1 type I collagen (COL1A1) gene, alpha-1 chain of type III collagen (COL3A1) gene, transforming growth factor beta 1 (TGF-β1) gene, actin alpha 2, smooth muscle (ACTA2) gene, and TIMP metallopeptidase inhibitor 1 (TIMP1) gene.

10. The health functional food composition of claim 6, wherein the Sanguisorba officinalis extract is included at 1 to 100 μg/mL in the composition, and the ziyuglycoside is included at 1 to 100 μM in the composition.


SEQUENCE LISTING (28 sequences)

SEQ ID NO: 1 - DNA, 19 bp, synthetic construct
  source 1..19: mol_type = other DNA; note = COL1A1_F
atggagctcctggtcagat

SEQ ID NO: 2 - DNA, 20 bp, synthetic construct
  source 1..20: mol_type = other DNA; note = COL1A1_R
gtagcaccatcatttccacg

SEQ ID NO: 3 - DNA, 19 bp, synthetic construct
  source 1..19: mol_type = other DNA; note = COL3A1_F
gctctgcttcatcccacta

SEQ ID NO: 4 - DNA, 20 bp, synthetic construct
  source 1..20: mol_type = other DNA; note = COL3A1_R
cgcataggactgaccaagat

SEQ ID NO: 5 - DNA, 18 bp, synthetic construct
  source 1..18: mol_type = other DNA; note = ACTA2_F
gccaagcactgtcaggaa

SEQ ID NO: 6 - DNA, 19 bp, synthetic construct
  source 1..19: mol_type = other DNA; note = ACTA2_R
attgtcacacaccaaggca

SEQ ID NO: 7 - DNA, 21 bp, synthetic construct
  source 1..21: mol_type = other DNA; note = TIMP1_F
ctctgaaaagggcttccagtc

SEQ ID NO: 8 - DNA, 21 bp, synthetic construct
  source 1..21: mol_type = other DNA; note = TIMP1_R
aggattcaggctatctgggac

SEQ ID NO: 9 - DNA, 20 bp, synthetic construct
  source 1..20: mol_type = other DNA; note = GAPDH_F
cagccgcatcttcttttgcg

SEQ ID NO: 10 - DNA, 20 bp, synthetic construct
  source 1..20: mol_type = other DNA; note = GAPDH_R
tccgttgactccgaccttca

SEQ ID NO: 11 - DNA, 20 bp, synthetic construct
  source 1..20: mol_type = other DNA; note = Ppara_F
gaactgacgtttgtggctgg

SEQ ID NO: 12 - DNA, 20 bp, synthetic construct
  source 1..20: mol_type = other DNA; note = Ppara_R
gctctctgtgtccaccatgt

SEQ ID NO: 13 - DNA, 20 bp, synthetic construct
  source 1..20: mol_type = other DNA; note = Lpl_F
gtggacatcggagaactgct

SEQ ID NO: 14 - DNA, 20 bp, synthetic construct
  source 1..20: mol_type = other DNA; note = Lpl_R
cctctcgatgacgaagctgg

SEQ ID NO: 15 - DNA, 23 bp, synthetic construct
  source 1..23: mol_type = other DNA; note = b-actin_F
cattgctgacaggatgcagaagg

SEQ ID NO: 16 - DNA, 22 bp, synthetic construct
  source 1..22: mol_type = other DNA; note = b-actin_R
tgctggaaggtggacagtgagg

SEQ ID NO: 17 - DNA, 20 bp, synthetic construct
  source 1..20: mol_type = other DNA; note = Sod1_F
gggaagcatggcgatgaaag

SEQ ID NO: 18 - DNA, 20 bp, synthetic construct
  source 1..20: mol_type = other DNA; note = Sod1_R
gccttctgctcgaagtggat

SEQ ID NO: 19 - DNA, 20 bp, synthetic construct
  source 1..20: mol_type = other DNA; note = Cat_F
caagattgccttctccgggt

SEQ ID NO: 20 - DNA, 20 bp, synthetic construct
  source 1..20: mol_type = other DNA; note = Cat_R
atggtgtaggattgcggagc

SEQ ID NO: 21 - DNA, 20 bp, synthetic construct
  source 1..20: mol_type = other DNA; note = Gpx1_F
agtccaccgtgtatgccttc

SEQ ID NO: 22 - DNA, 20 bp, synthetic construct
  source 1..20: mol_type = other DNA; note = Gpx1_R
cctcagagagacgcgacatt

SEQ ID NO: 23 - DNA, 20 bp, synthetic construct
  source 1..20: mol_type = other DNA; note = Col1a1_F
agcacgtctggtttggagag

SEQ ID NO: 24 - DNA, 20 bp, synthetic construct
  source 1..20: mol_type = other DNA; note = Col1a1_R
gacattaggcgcaggaaggt

SEQ ID NO: 25 - DNA, 20 bp, synthetic construct
  source 1..20: mol_type = other DNA; note = Col3a1_F
gtggacattggccctgtttg

SEQ ID NO: 26 - DNA, 20 bp, synthetic construct
  source 1..20: mol_type = other DNA; note = Col3a1_R
agttggtcacttgcactggt

SEQ ID NO: 27 - DNA, 20 bp, synthetic construct
  source 1..20: mol_type = other DNA; note = Tgf-b1_F
gtggctgaaccaaggagacg

SEQ ID NO: 28 - DNA, 20 bp, synthetic construct
  source 1..20: mol_type = other DNA; note = Tgf-b1_R
gtttggggctgatcccgttg